(19) United States
(12) Patent Application Publication
(10) Pub. No.: US 2024/0279282 A1
(43) Pub. Date: Aug. 22, 2024

(54) SELF-ASSEMBLED CYCLIC PEPTIDE NANOTUBES FOR LIGHT ACTUATION

(71) Applicant: The Government of the United States of America, as represented by the Secretary of the Navy, Arlington, VA (US)

(72) Inventors: Kenan Fears, Alexandria, VA (US); Manoj K. Kolel-Veetil, Annandale, VA (US); Olufolasade Atoyebi, Washington, DC (US); William A. Maza, Silver Spring, MD (US)

(73) Assignee: The Government of the United States of America, as represented by the Secretary of the Navy, Arlington, VA (US)

(21) Appl. No.: 18/433,043

(22) Filed: Feb. 5, 2024

Related U.S. Application Data
(60) Provisional application No. 63/483,054, filed on Feb. 3, 2023.

(51) Int. Cl. C07K 7/64; C07K 5/12; C08G 73/10

(52) U.S. Cl. CPC C07K 7/64; C07K 5/126; C08G 73/1096

(57) ABSTRACT

Disclosed is a cyclic peptide polymer having the below structure. Each R¹ and each R² is an organic group. Each R³ is a covalent bond or methylene. The values m and n are nonnegative integers having a sum of at least 1. Each X is —NH—CO— or —CO—N—. The value p is an integer greater than 1. The cyclic peptide polymer may be made by providing a cyclic peptide having two side chains having terminal amino groups or carboxylic acid groups, and reacting the amino groups with an azobenzene or the carboxylic acid groups with an azodianiline.

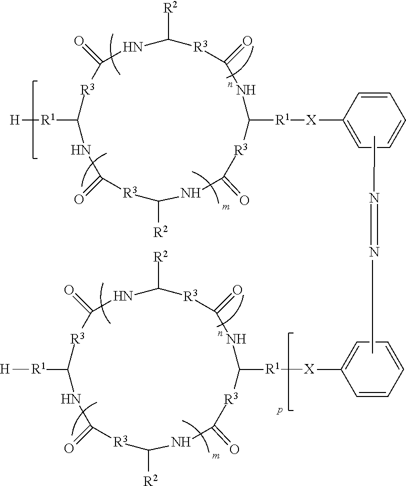

### REFERENCE TO RELATED APPLICATIONS

This application claims the benefit of U.S. Provisional Application No. 63/483,054, filed on Feb. 3, 2023. The provisional application and all other publications and patent documents referred to throughout this nonprovisional application are incorporated herein by reference.

### TECHNICAL FIELD

The present disclosure is generally related to light-actuated cyclic peptide polymers.

### DESCRIPTION OF THE RELATED ART

Using carbon nanotubes as inspiration, DeSantis et al. theorized and then Ghadiri et al. successfully synthesized a bio-inspired peptide nanotube (Ghadiri et al. Nature, 1993, 366, 324-327). These are flat hollow rings that stack and interact through hydrogen bonding emanating from the rings' backbone, while side chains along the plane of the ring point away from the rings' center. Rubin et al. experimentally studied the mechanical properties of PNP and found that while they demonstrate a stiffness and strength like that of bone, the extensibility of the PNP was an order of magnitude less (Rubin et al. ACS Nano 2015, 9, 3360-3368). Fears et al. posited, and later observed experimentally, that covalently linking the rings to synthesize a cyclic peptide polymer chain, improved the extensibility of the cyclic peptide nanotube while maintaining its ideal/innate strength and pH-responsive self-assembly (U.S. Pat. No. 8,975,368; Fears et al. Nature Comm. 2018, 9, 4090). Other stimuli have also been used in conjunction with cyclic peptides to induce a conformational change. Renner et al. incorporated azobenzene within a unit/component of a cycle formed by the constituent peptide monomers and reported the azobenzene as a photo-responsive conformational switch in cyclic peptides (Renner et al. J. Peptide Res. 2005, 65, 4-14). They reported the formation of mono- and bicyclic peptides that contained azobenzene and demonstrated the conformational changes of these structures caused by the photoisomerization of azobenzene. However, no assembling of the azobenzene-containing cyclic peptide unit to a nanotubular structure was reported. A similar strategy was also used by Mart and Allemann wherein an azobenzene moiety contained in a cyclic peptide unit further caused the coiling/‘helix’ing of the cyclic unit (Mart et al. Chem. Commun. 2016, 52, 12262). Again, as with Renner et al.'s case, there was no formation of a cyclic peptide nanotube in this case. Recently, Novelli et al. reported the light-induced molecular piping of cyclic peptide nanotubes to form bent tubular structures (Novelli et al. Angew. Chem. Int. Ed. 2021, 60, 18838-18844). The process was based on the [4+4] photocycloaddition of anthracene moieties, whose structural changes derived from the interdigitated flat disposition of precursors to the corresponding cycloadduct moieties, induced the geometrical modifications in nanotubes packing that caused their curvature. The new class of cyclic peptide nanotubes were formed from β- and α-amino acids. The β-amino acid groups predisposed the cyclic peptide rings to stack in parallel fashion with the β-amino acid groups aligned along the nanotube with a homogeneous distribution of anthracene pendant entities. When nanotubes with anthracene pedant entities came together, the entities formed an interdigitated structure, which upon photoexcitation caused the nanotubes to bend through photocycloaddition. In this example, it is noteworthy that there is no covalent linking between individual cyclic peptide rings of a singular cyclic peptide nanotube; but the linking due to stacking and interdigitation of the anthracene entities occurs between two distinct nanotubes.

### SUMMARY OF THE INVENTION

Disclosed herein is a cyclic peptide polymer having the below structure. Each R¹ and each R² is an independently selected organic group. Each R³ is independently selected from covalent bond and methylene. The values m and n are nonnegative integers having a sum of at least 1. Each X is independently selected from —NH—CO— and —CO—N—. The value p is an integer greater than 1.

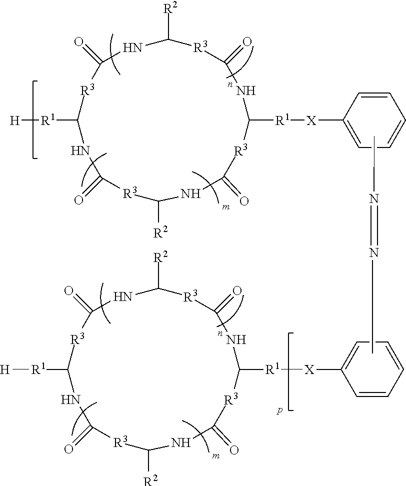

Also disclosed herein is a method comprising: providing a cyclic peptide comprising two side chains having terminal amino groups or carboxylic acid groups, and reacting the amino groups with an azobenzene or the carboxylic acid groups with an azodianiline to form a cyclic peptide polymer.

### BRIEF DESCRIPTION OF DRAWINGS

A more complete appreciation will be readily obtained by reference to the following Description of the Example Embodiments and the accompanying drawings.

FIG. 1 shows two adjacent cyclic peptide units covalently linked by an azobenzene undergoing self-assembly modification due to isomerization of azobenzene either due to light or heat.

FIG. 2 shows a schematic of a chain of peptide units covalently linked by azobenzene units undergoing self-assembly modification due to isomerization of azobenzene due to light.

FIG. 3 shows a synthetic scheme for a cyclic peptide monomer.

FIG. 4 shows a synthetic scheme for a cyclic peptide polymer.

FIG. 5A shows an AFM image of the trans structure of the azobenzene-linked polymerized cyclic α-D,L-KVVKVV peptide.

FIG. 5B shows a UV-Vis spectra of the trans structure of the azobenzene-linked polymerized cyclic α-KvVkVv peptide.

FIG. 6A shows an AFM image of the cis structure of the azobenzene-linked polymerized cyclic α-KvVkVv peptide.

FIG. 6B shows a UV-vis spectrum of the cis structure of the azobenzene-linked polymerized cyclic α-KvVkVv peptide.

FIG. 7 shows UV-vis measurements of the thermal relaxation of the azobenzene-linked polymerized cyclic α-KvVkVv peptide after photolysis.

FIG. 8 shows time-dependent absorbance measurements of the thermal relaxation of the azobenzene-linked polymerized cyclic α-KvVkVv peptide after photolysis.

FIG. 9 shows absorbance measurements of the polymerized cyclic peptide after repeated exposure to visible and UV light.

FIG. 10 shows the corresponding UV-vis spectrum of the polymerized cyclic peptide.

FIG. 11 shows the change in absorbance at 314 nm after UV-irradiation at two temperatures.

### DETAILED DESCRIPTION

In the following description, for purposes of explanation and not limitation, specific details are set forth in order to provide a thorough understanding of the present disclosure. However, it will be apparent to one skilled in the art that the present subject matter may be practiced in other embodiments that depart from these specific details. In other instances, detailed descriptions of well-known methods and devices are omitted so as to not obscure the present disclosure with unnecessary detail.

Disclosed herein is a method of controlling the self-assembly of a cyclic peptide nanotube through a chosen crosslinker. Using azobenzene or azodianiline as the crosslinker to polymerize the cyclic peptides, this method capitalizes on the linker's ability to change its conformation, due to photo-isomerization, and thus leading to the self-assembly of the peptide nanotube polymer (PNP). Importantly, this is not limited to controlling self-assembly of the PNP through azobenzene alone, or any other light-actuable crosslinker, but encompasses self-assembly of the cyclic peptide nanotube through conducive crosslinkers that have various materials properties including a stimuli-responsive nature.

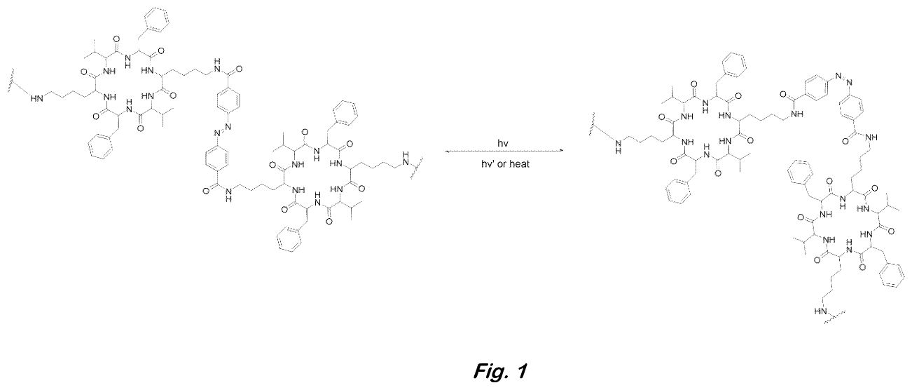

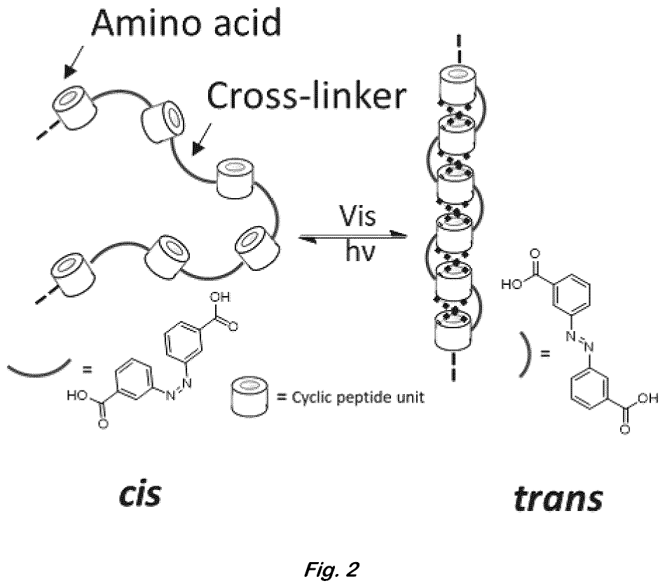

In the presently disclosed method, the polymerized cyclic peptide may be synthesized by covalently linking the rings of a singular cyclic peptide polymer with azobenzene (FIGS. 1 and 2). Self-assembly into a PNP, i.e. nanotube, occurs via the linker, in the presence of ultraviolet light/visible light, and potentially due to temperature because of a transition from trans- to cis-form of the azobenzene. In U.S. Pat. No. 8,975,368, the assembly of PNPs was controlled by manipulating the electrostatic charge of the amino acid residues in the peptide rings through the pH of the solution. Whether the peptides were covalently linked or not, each peptide ring is capable of stacking and interacting with one another through hydrogen bonding via the amide bonds in the rings' backbone. However, pH-responsive assembly of the peptide nanotubes is dependent on the presence of amino acids susceptible to protonation or deprotonation on the ring. In the present method, the peptide assembly is dependent on the linker and not the amino acids present in the ring. Experimentally, the rings are covalently linked using azobenzene, a photo-isomeric compound, and self-assembly of the PNPs is controlled photochemically. After polymerization, the cyclic peptide polymer appears to be in the unfolded state, where the azobenzene is in its trans conformation. In the presence of ultraviolet (UV) light, the azobenzene changes from the trans conformation to the cis conformation, assembling the peptide nanotube. Furthermore, linker dependent self-assembly is not limited to photosensitive materials but could include thermally responsive linkers, pH-responsive linkers, and other stimuli-responsive linkers. Depending on the linker used, peptide nanotube self-assembly could be stimuli-responsive, adding a variety of potential uses to the peptide nanotubes.

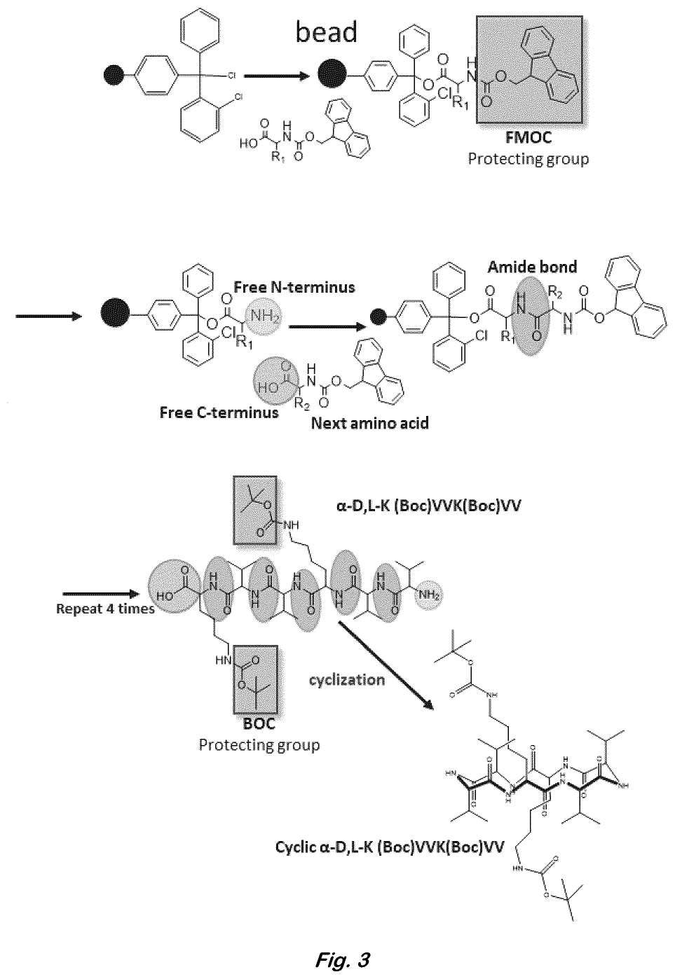

The polymer is made from a cyclic peptide monomer. Such monomers may be made, for example, as disclosed in U.S. Pat. Nos. 8,975,368 and 11,447,524. FIG. 3 shows a synthetic scheme for a cyclic peptide monomer, where an amino acid having a protecting group is bound to a solid support and the protecting group removed. This is followed by successive additions of protected amino acid and removal of the protecting group. Finally, the linear peptide is cleaved from the support and cyclized. The side chains to be reacted with azobenzene or azodianiline may have protecting groups that are removed before cyclization.

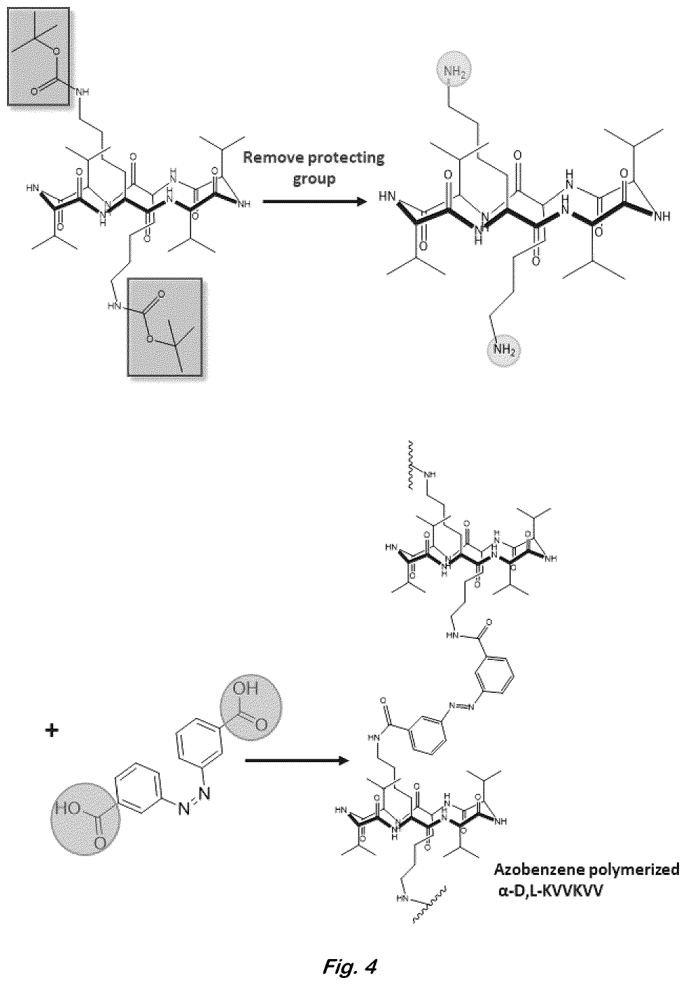

The amino acids in the cyclic peptide may be, for example, a or ß amino acids, and may have D or L chirality. The side chains of the amino acids should include at least two carboxylic acid groups, at least two amino groups, or at least one of each. FIG. 4 shows a synthetic scheme for a cyclic peptide polymer having two amino groups, which are reacted with the carboxylic acid groups of azobenzene. When the cyclic peptide monomer has carboxylic acid groups, they may be reacted with the amino group of azodianiline. Further, azobenzene, azodianiline, and pendant carboxylic acid and amino groups may be used in combination.

The resulting cyclic peptide polymer may be repeatedly converted between nanotube form and random coil form by application of appropriate light or heat stimulus to convert the azobenzene or azodianiline between cis and trans forms. This method is a way of self-assembling the polymerized cyclic peptides using characteristics from the desired linker to control self-assembly in addition to the rings' electrostatic interactions with each other. This may provide certain advantages: 1) Using stimuli-responsive compounds, such as photo-actuable azobenzene, to covalently link the cyclic peptides, can provide the nanotubes with additional characteristics and different methods to control self-assembly. This could lead to the production of thermo-sensitive self-assembling peptide nanotubes, and other stimuli-responsive self-assembling peptide nanotubes. 2) As a result of shifting self-assembly of the peptide nanotube from the ring to the linker, this allows for more variety in the amino acids chosen to make up the cyclic peptide. Using different amino acids could change the potential use of the peptide nanotubes.

The following examples are given to illustrate specific applications. These specific examples are not intended to limit the scope of the disclosure in this application.

### Example 1—Synthesis of Cyclic Peptides

Linear peptides were synthesized using a Liberty Blue HT12 automated microwave-assisted peptide synthesizer (CEM). Two peptide sequences were synthesized consisting of either α- or β-amino acids, bearing the same sequence of amino acid side chains. The linear sequence of α-KvVkVv was synthesized using Fmoc-α-Lys(Boc)-OH (K), Fmoc-α-D-Val-OH (v), Fmoc-α-Val-OH (V), and Fmoc-α-D-Lys(Boc)-OH (k). The linear sequence of β-KVVKVV was synthesized using Fmoc-β-HomoLys(Boc)-OH (K) and Fmoc-β-Homo Val-OH (V). All amino acids were obtained from Chemimpex (Wood Dale, IL). Solid phase peptides synthesis was performed on acid labile resin, 2-chlorotrityl chloride. Amino acid coupling and deprotection reactions were carried out at 60° ° C. for 5 or 15 minutes, respectively, using N,N′-diisopropylcarbodiimide (DIC) as the activator, ethyl cyanohydroxyiminoacetate (Oxyma) as the activator base, and 20% piperidine in DMF as the de-protection cocktail. Upon completion of peptide synthesis, linear peptides were cleaved from using 2% trifluoroacetic acid (TFA) in dichloromethane (DCM), resulting in linear peptides with Boc protecting groups in place. Linear peptides were purified by cold ether precipitation. Molecular weights were verified using matrix assisted laser desorption ionization-time of flight mass spectrometry (MALDI-TOF MS) using α-cyano-4-hydroxycinnamic acid (CHCA) as the matrix substance. The yields of linear α-KvVkVv and β-KVVKVV based on the weight of crude material were 95% and 82%, respectively.

Linear peptides were cyclized in dilute solutions of 20% N-methylmorpholine (NMM) in DMF (5 mM) with 12 equivalents of DIC and 12 equivalents of Oxyma. Cyclization reactions were carried out on a Discover 2.0 microwave synthesizer (CEM) at 90° C. for 20 min. Upon completion of cyclization reactions, solutions were dried on a rotary evaporator. Cyclized peptides were fully de-protected using a solution consisting of 95% TFA, 2.5% water, and 2.5% triisopropyl silane (TIPS). Peptides were purified by cold ether precipitation followed by reverse-phase high performance liquid chromatography (RP-HPLC) with a C18 column and molecular weights were verified with MALDI-TOF MS. The total yields of cyclic α-KvVkVv and β-KVVKVV were 87% and 75%, respectively.

### Example 2—Synthesis of Polymerized Cyclic Peptides

Polymers were formed by coupling purified cyclic peptides with 3,3′-azodibenzoic acid or 4,4′-azodibenzoic acid in a 1:1 molar ratio. Concentrated peptide solutions (0.2 M) were prepared in 20% NMM in DMF with 12 equivalents of DIC and 12 equivalents of Oxyma. Polymerization reactions were carried out on a Discover 2.0 microwave synthesizer (CEM) at 90° C. for 2 hours. The resulting polymers were purified by RP-HPLC with a silica or size exclusion column. Final yields for the α- and β-polymers were 80% and 65%, respectively, and molecular weights were ca. 80 kDa.

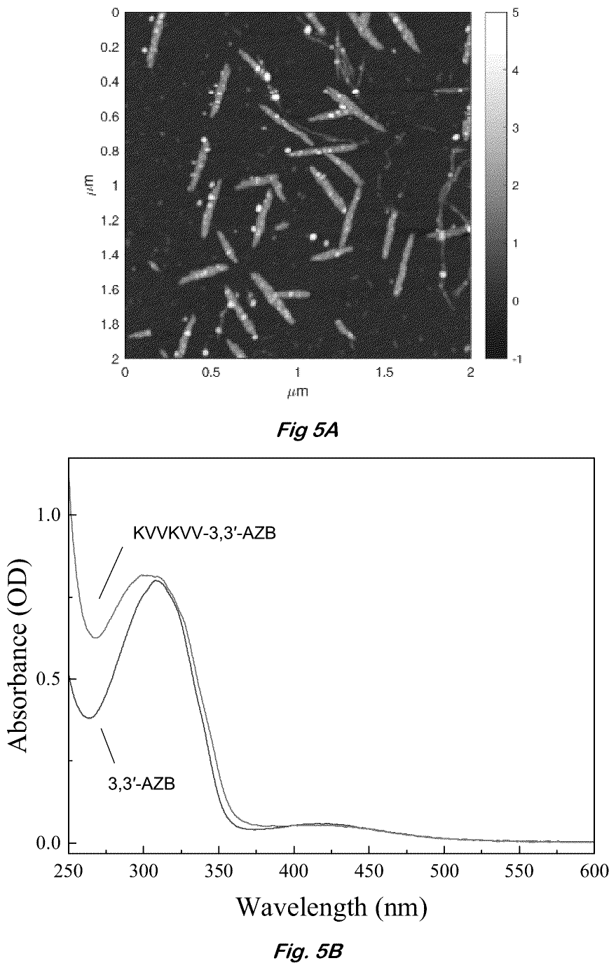

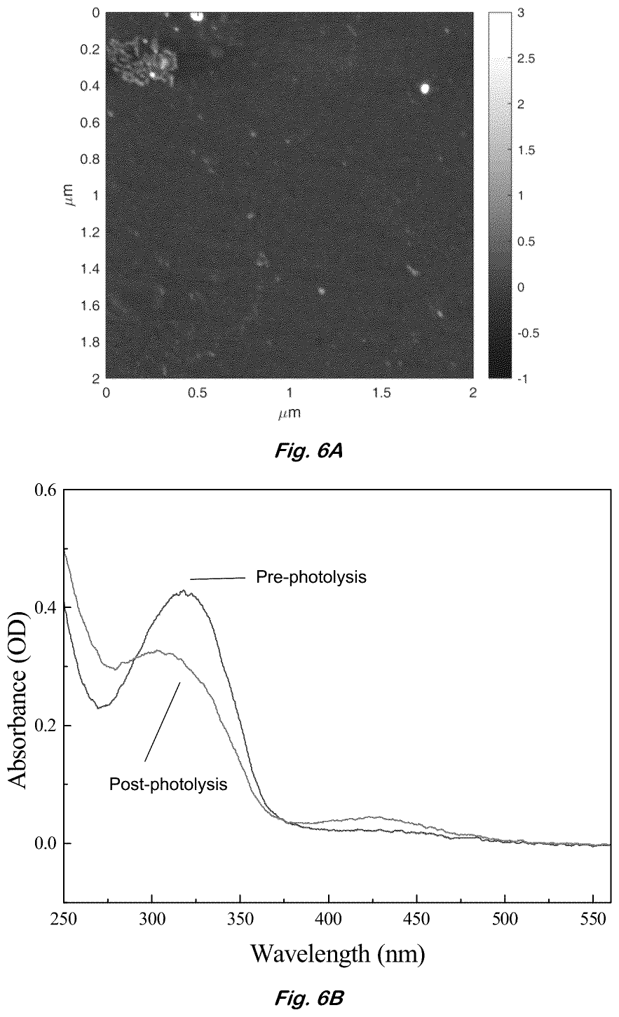

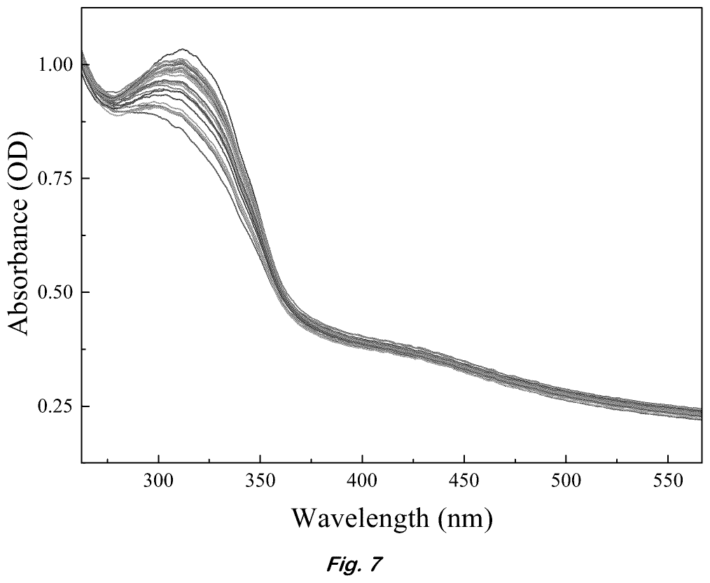

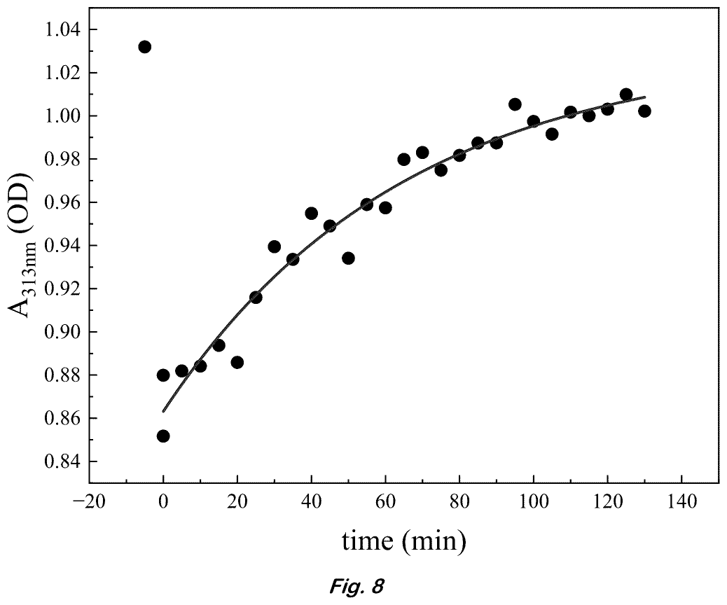

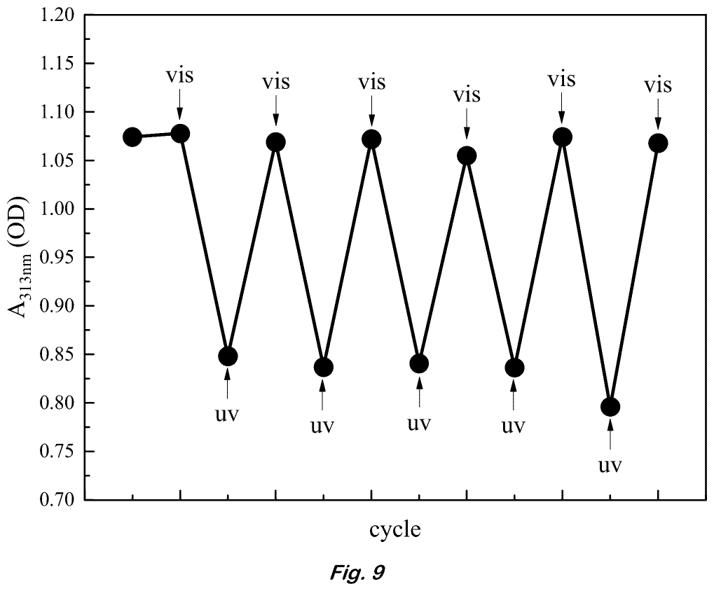

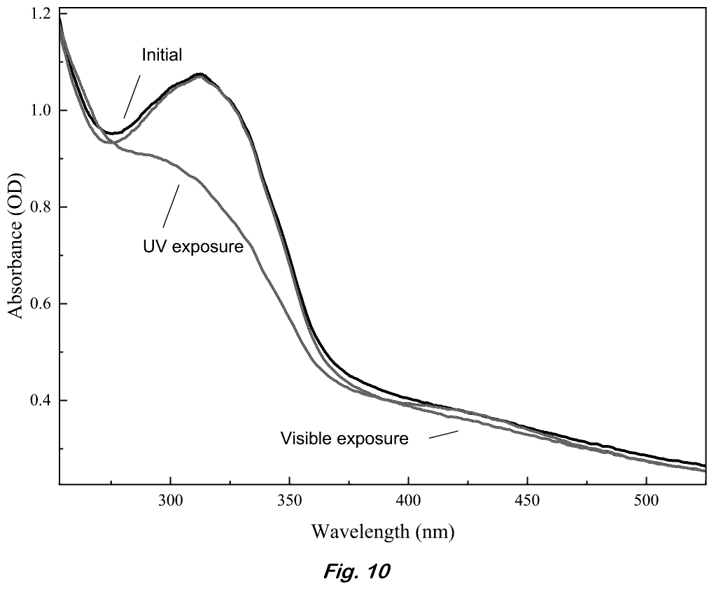

FIGS. 5A and 6A show AFM images of the nanotube and random coil forms of the α-KvVkVv polymer, with their UV-vis spectra shown in FIGS. 5B and 6B. Further characterization of the polymer are shown in FIGS. 7-10, including UV-vis measurements of the thermal relaxation after photolysis (FIG. 7), time-dependent absorbance measurements of the thermal relaxation after photolysis (FIG. 8), absorbance measurements after repeated exposure to visible and UV light (FIG. 9), and the corresponding UV-vis spectrum (FIG. 10).

### Example 3—Temperature Stimulus

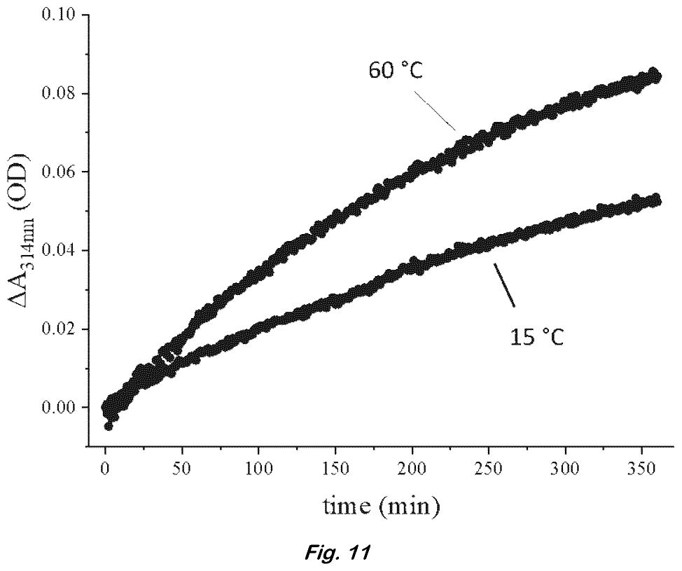

The rate of the cis-to-trans thermal isomerization of the azobenzene-linked α-KVVKVV cyclic peptide was monitored by following the change in absorbance at 314 nm after UV-irradiation (FIG. 11). Solutions of the cyclic peptide were prepared in trifluoroethanol such that the optical density at the absorbance maximum ˜310 nm was ˜0.5. The capped sample was irradiated using a 310 nm LED for 30 min with continuous magnetic stirring. The sample temperature was modulated using a temperature-controlled cuvette holder and was stable to +1° C.

No significant changes were observed in the position of the 310 nm absorbance maximum of the equilibrium spectrum of the trans-isomer of the azobenzene-linked cyclic peptide as a function of temperature between 15° C. and 60° C. Upon UV-induced photoisomerization, the cis-to-trans thermal relaxation increases nearly two-fold upon increasing the solution temperature from 15° C. to 60° C. The results suggest that the thermally activated cis-to-trans isomerization of the azobenzene has a small activation energy (<1KJ/mol).

Many modifications and variations are possible in light of the above teachings. It is therefore to be understood that the claimed subject matter may be practiced otherwise than as specifically described. Any reference to claim elements in the singular, e.g., using the articles “a”, “an”, “the”, or “said” is not construed as limiting the element to the singular.

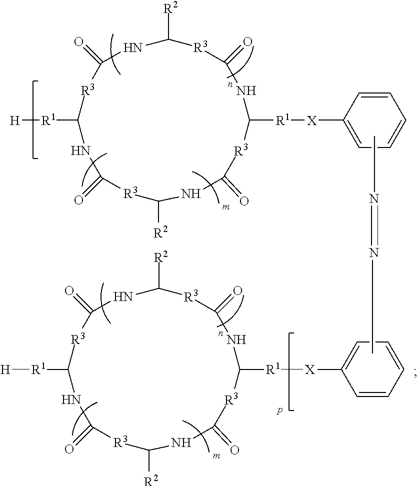

wherein each R¹ and each R₂ is an independently selected organic group;
wherein each R³ is independently selected from covalent bond and methylene;
wherein m and n are nonnegative integers having a sum of at least 1;
wherein each X is independently selected from —NH—CO— and —CO—N—; and
wherein p is an integer greater than 1.

2. The cyclic peptide polymer of claim 1, wherein the cyclic peptide polymer has the structure:

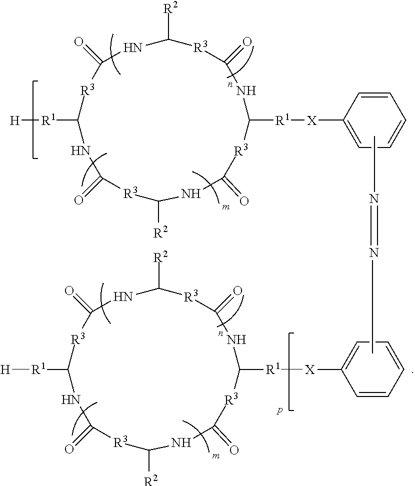

3. The cyclic peptide polymer of claim 2, wherein the cyclic peptide polymer has the structure:

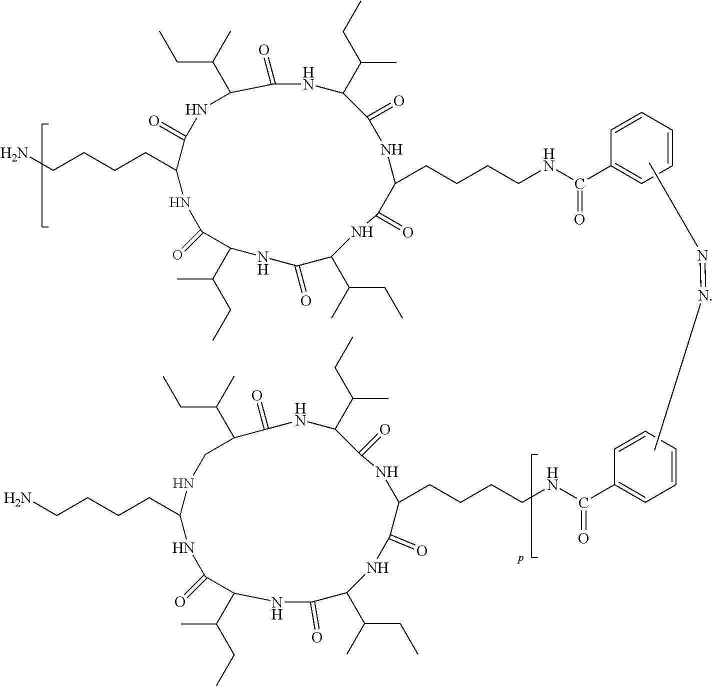

4. The cyclic peptide polymer of claim 2, wherein the cyclic peptide polymer has the structure:

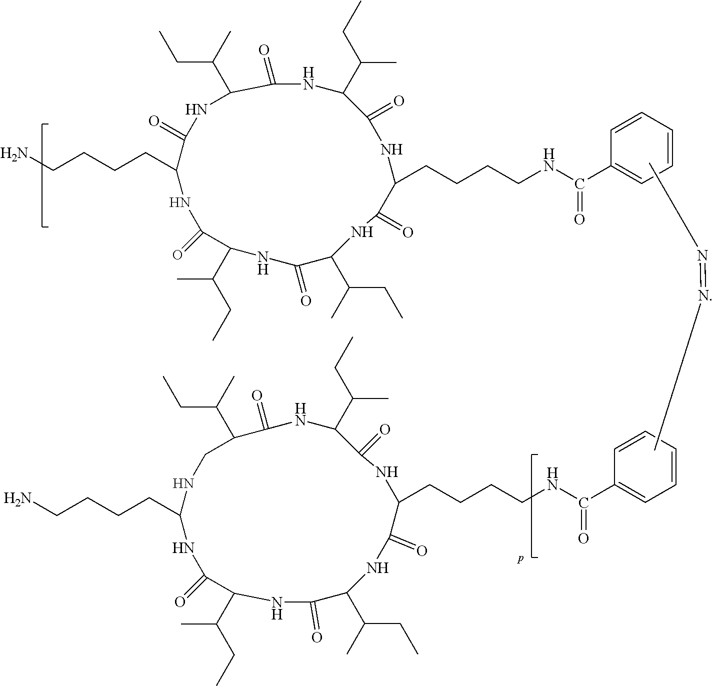

5. The cyclic peptide polymer of claim 1, wherein the cyclic peptide polymer has the structure:

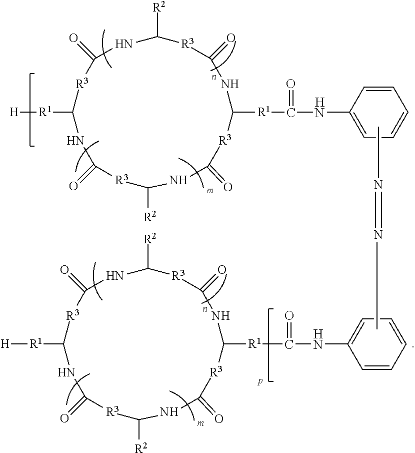

6. A method comprising:
providing the cyclic peptide polymer of claim 1;
wherein the —N≡N— bond is in either a cis state or a trans state;
exposing the cyclic peptide polymer to light that converts the cis state to the trans state or the trans state to the cis state.

7. A method comprising:
providing a cyclic peptide monomer comprising two side chains having terminal amino groups or carboxylic acid groups;
reacting the amino groups with an azobenzene or the carboxylic acid groups with an azodianiline to form a cyclic peptide polymer.
